(19) United States
(12) Patent Application Publication
(10) Pub. No.: US 2006/0264423 A1
(43) Pub. Date: Nov. 23, 2006

(54) Methylene Blue Therapy of Viral Disease

(75) Inventors: Christopher Wood, Stoke Poges (GB); Nagy Habib, Cairo (EG)

Correspondence Address: PATREA L. PABST;PABST PATENT GROUP LLP, 400 COLONY SQUARE, SUITE 1200, ATLANTA, GA, 30361, US

(73) Assignee: BIOENVISION, INC.

(21) Appl. No.: 11/419,426

(22) Filed: May 19, 2006

Related U.S. Application Data
(60) Provisional application No. 60/682,891, filed on May 20, 2005.
(60) Provisional application No. 60/718,804, filed on Sep. 20, 2005.
(60) Provisional application No. 60/697,094, filed on Jul. 7, 2005.
(60) Provisional application No. 60/720,066, filed on Sep. 23, 2005.
(60) Provisional application No. 60/720,147, filed on Sep. 23, 2005.
(60) Provisional application No. 60/720,058, filed on Sep. 23, 2005.

(51) Int. Cl. A61K 31/5415; A61K 31/538

(52) U.S. Cl. 514/224.8; 514/229.8

(57) ABSTRACT

A method for using thiazine dyes, especially methylene blue or methylene blue derivatives, in an immediate or controlled release formulation, alone or in combination with low levels of light or other drugs, to selectively inactivate or inhibit hepatitis infection, has been developed. Clinical trial results demonstrate efficacy in a human clinical trial for treatment of hepatitis C by oral administration of methylene blue immediate release formulation, in a dosage of 65 mg twice daily, over a period of at least 100 days. A method for using thiazine dyes, especially methylene blue or methylene blue derivatives, in an immediate or controlled release formulation, along or in combination with low levels of light or other drugs, to prevent or decrease reactivation of viruses, is also described. The preferred class of patient is infected with, or has been exposed to, viruses such as Herpes simplex virus type 1 & 2, Varicella zoster virus, Epstein-Barr virus, Cytomegalovirus, and Herpes virus type 6 & 7, Adenovirus, and Human polyoma viruses, e.g. JC virus and BK virus. In one embodiment the thiazine dye is administered to a patient experiencing symptoms or disease caused by reactivation of a virus. In a preferred embodiment the thiazine dye is administered to a patient at risk for or experiencing symptoms or disease caused by reactivation of a virus, prior to or during immunosuppression or chemotherapy.

### BACKGROUND OF THE INVENTION

This application claims priority to U.S. Ser. No. 60/682,891 filed May 20, 2005; U.S. Ser. No. 60/718,804 filed Sep. 20, 2005; U.S. Ser. No. 60/697,094 filed Jul. 7, 2005; U.S. Ser. No. 60/720,066 filed Sep. 23, 2005; and U.S. Ser. No. 60/720,058 filed Sep. 23, 2005.

This invention is generally in the area of methods for the treatment of viral diseases, and more specifically relates to the treatment of hepatitis virus using thiazine dyes, and in particular methylene blue, and to the treatment of reactivated viruses using thiazine dyes, and in particular methylene blue.

Hepatitis C Virus (“HCV”) enters the body through direct blood exposure. The virus attacks cells in the liver, where it multiplies (replicates). HCV causes liver inflammation and kills liver cells. After exposure to the virus, the incubation period usually last 2-26 weeks. The initial phase of acute infection usually resolves after 2-12 weeks. However, up to 80-85% of people initially infected with HCV do not clear the virus from their bodies, and become chronically infected. Most people with chronic HCV do not have symptoms and lead relatively normal lives. But in 10-25% of people, the disease progresses over the course of 10-40 years. Chronic HCV infection can lead to liver damage, the development of fibrous tissue in the liver, fat deposits in the liver (steatosis), cirrhosis of the liver, and liver cancer. Liver cancer usually develops at later stages of HCV infection, typically after 25-30 years. The type of liver cancer associated with HCV is called primary hepatocellular carcinoma (HCC). Today, HCV is the leading cause of liver transplants.

There is currently no vaccine or cure for HCV, but various treatments can reduce or stop virus replication and help slow or stop disease progression. The treatment of choice today is a combination of pegylated interferon-alpha2a (“peginterferon”) and ribavirin. In a recent randomized, double-blind trial designed to assess the efficacy and safety of 24 or 48 weeks of treatment with peginterferon-alpha2a plus a low or standard dose of ribavirin, a total of 1311 patients with chronic hepatitis were studied. Overall and in patients infected with HCV genotype 1, 48 weeks of treatment was statistically superior to 24 weeks and standard-dose ribavirin was statistically superior to low-dose ribavirin. In patients with HCV genotype 1, absolute differences in sustained virologic response rates between 48 and 24 weeks of treatment were 11.2% (95% CI, 3.6% to 18.9%) and 11.9% (CI, 4.7% to 18.9%), respectively, between standard- and low-dose ribavirin. Sustained virologic response rates for peginterferon-alpha2a and standard-dose ribavirin for 48 weeks were 64% (CI, 59% to 68%) overall and 52% (CI, 46% to 58%) in patients with HCV genotype 1. In patients with HCV genotypes 2 or 3 , the sustained virologic response rates in the 4 treatment groups were not statistically significantly different. The study concluded that treatment with peginterferon-alpha2a and ribavirin may be individualized by genotype. Patients with HCV genotype 1 require treatment for 48 weeks and a standard dose of ribavirin; those with HCV genotypes 2 or 3 appear to be adequately treated with a low dose of ribavirin for 24 weeks.

Unfortunately, even if these therapies are effective, the cost is prohibitive in many countries such as Egypt.

It is therefore and object of the present invention to provide methods and compositions for treatment or prevention of hepatitis infections.

It is a further object of the present invention to provide methods and compositions for relatively inexpensive treatment of hepatitis infections, especially hepatitis C infection.

Most DNA virus infections are reactivation-infection, i.e. infections caused by reactivation of the virus that has been dormant after the initial, primary infection months to many years prior to the reactivation. Reactivation can occur when the immune system loses control of the latent virus allowing it to become active. The loss of control occurs when some event or illness results in depressed or suppressed immunity (even if temporary). Examples of such events are acute infection with other viruses (influenza, HIV, etc.), infectious mononucleosis, nerve trauma, physiologic and physical changes (e.g., fever menstruation, and sunlight, immunosuppression, surgery, trauma, toxic exposure, chemotherapy, radiation exposure, extreme acute stress and chronic stress.

The antiviral compounds acyclovir, valaciclovir and famciclovir are presently available for the treatment of HSV disease. Prophylactic intravenous or oral acyclovir has become a standard of care for HSV seropositive cancer patients during period of profound immunosuppression. Allogeneic stem cell transplant (SCT) recipients also develop acute graft-versus-hose disease usually require a prolonged HSV prophylaxis. Valaciclovir and famciclovir have an oral bioavailability 3-5 times superior to that or oral acyclovir. Although not systematically studied, oral valaciclovir is commonly used in the prevention of HSV reactivation after SCT. Intravenous acyclovir remains the therapy of chose for severe mucocutaneous or visceral HSV disease in immunocompromised hosts. Despite these treatments, it has been demonstrated that for patients receiving haploidentical SCT, which required stringent T- and B-cell depletion of the graft to prevent fatal graft-versus-hose disease (GVHD), reactivation of latent human herpes viruses occurred in all 7 evaluable HSV-1—or HSV-2-seropositive patients and those patients did not respond to acyclovir treatment (Langston, A., et al., Blood 99(3):1085-1088 (2002). In addition, these drugs can loose their effectiveness over time, have low solubility, and do not always prevent outbreaks of the virus. The most common adverse effects of these antiviral drugs include nausea, headache, vomiting, dizziness, and abdominal pain.

Given the toxicity associated with current antiviral agents, the cost and implementation difficulties, and the frequent occurrence of resistant viral strains to antivirals, it is apparent that inexpensive, safe, and highly effective treatments for reactivation of viral infections is needed, especially in patients who are also immunosuppressed.

It is therefore an additional object of the present invention to provide methods and compositions for treatment or prevention of reactivation of viral infections.

It is further an object of the present invention to provide safe and effective methods and compositions for treatment or prevention of reactivation of viral infections in patients who are immunosuppressed, e.g. undergoing chemotherapy.

It is a further object of the present invention to provide methods and compositions for relatively inexpensive treatment of reactivation of viruses.

### SUMMARY OF THE INVENTION

A method for using thiazine dyes, especially methylene blue, methylene blue derivatives, or pharmaceutically acceptable salts thereof, in an immediate or controlled release formulation, alone or in combination with low levels of light or other drugs, to selectively inactivate or inhibit hepatitis infection, has been developed. Clinical trial results demonstrate efficacy in a human clinical trial for treatment of hepatitis C by oral administration of methylene blue immediate release formulation, in a dosage of 65 mg twice daily, over a period of at lease 100 days.

A method for using thiazine dyes, especially methylene blue, methylene blue derivatives, or pharmaceutically acceptable salts thereof, in an immediate or controlled release formulation, along or in combination with low levels of light or other drugs, to prevent or decrease reactivation of viruses, is also described. The preferred class of patient is infected with, or has been exposed to, viruses such as Herpes simplex virus type 1 & 2, Varicella zoster virus, Epstein-Barr virus, Cytomegalovirus, and Herpes virus type 6 & 7, Adenovirus, and Human polyoma viruses, e.g. JC virus and BK virus. In one embodiment the thiazine dye is administered to a patient experiencing symptoms or disease caused by reactivation of a virus. In a preferred embodiment the thiazine dye is administered to a patient at risk for or experiencing symptoms or disease caused by reactivation of a virus, prior to or during immunosuppression or chemotherapy.

Examples of useful thiazine dyes are methylene blue, azure A, azure C, toluidine, and thionine. The preferred dye at this time is methylene blue. Since methylene blue absorbs in the red wavelengths, i.e., approximately 670 nm, which penetrates tissue much better than other lower wavelengths, light penetrating the skin to the capillaries at the surface can be used to enhance the activity of the dye. Pharmaceutical compositions comprising methylene blue, methylene blue derivatives, or pharmaceutically acceptable salts thereof are described herein. The compositions are preferably administered orally and can be administered as tablets, soft or hard shell capsules, suspension or solutions. The compositions further comprise a pharmaceutically acceptable carrier and optionally one or more pharmaceutically acceptable excipients. Suitable excipients include diluents, binders, plasticizers, lubricants, disintegrants, colorants, stabilizers, surfactants, and combinations thereof. The compositions can be formulated for controlled release of the active agent. Suitable controlled release formulations include delayed release, and combinations thereof.

The thiazine dye can be provided in combination with other known antibiotics, anti-inflammatories, antifungals, and antivirals.

### DETAILED DESCRIPTION OF THE INVENTION

I. Therapeutic Compositions

Pharmaceutical compositions for oral administration comprising methylene blue, a derivative thereof, or pharmaceutically acceptable salts thereof, in a pharmaceutically acceptable carrier, have been developed.

Definitions

Controlled release dosage form: A controlled release dosage form is one for which the drug release characteristics of time course and/or location are chosen to accomplish therapeutic or convenience objectives not offered by conventional dosage forms such as solutions, ointments, or promptly dissolving dosage forms. Delayed release, extended release, and pulsatile release forms and their combinations are types of controlled release dosage forms.

Delayed release dosage form: A delayed release dosage form is one that releases a drug (or drugs) at a time other than promptly after administration.

Extended release dosage form: An extended release dosage form is one that allows at lease a twofold reduction in dosing frequency as compared to that drug presented as a conventional dosage form (e.g. as a solution or prompt drug-releasing, conventional solid dosage form).

Pulsatile release dosage form: A pulsatile release dosage form is one that mimics a multiple dosing profile without repeated dosing and allows at lease a twofold reduction in dosing frequency as compared to that drug presented as a conventional dosage form. A pulsatile release profile is characterized by a time period of no release (lag time) followed by rapid drug release.

A. Methylene Blue And Its Derivatives

Dibenzo-1,4-thiazines, also known as phenothiazines, are a class of thiazine dyes which contain a six-membered heterocycle containing a single nitrogen atom and a single sulfur atom in which two benzene rings are fused to the heterocycle. Phenothiazine was first reported by Bernthsen in 1983 (Bernthsen, Ber. Deut. Chem. Ges., 16, 2896-2904, (1983).

Methylene blue is the most well known example of the phenothiazine dyes. The structure of methylene blue is shown below: 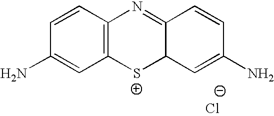
Methylene blue is used in a variety of applications such as textiles (for dyeing cellulosic fibers and printing leather), as an anti-oxidant and antiseptic, and in photogalvanic cells based on redox systems.

Methylene blue, or 3,7-Bis(dimethylamino)-phenothiazin-5-ium chloride, C₁₆H₁₈ClN₃S, is a dark green or blue thiazine dye which was first isolated in 1876. Methylene blue is a thiazine dye occurring as dark blue-green crystals which is soluble in water and sparingly soluble in alcohol, forming deep blue solutions. Methylene blue injectable has a pH of 3-4.5. The pK₃ is between 0 and −1.

Methylene blue and its analogues have also been used extensively for staining live and fixed tissues. Derivatives such Azure A, B, and C as well as Taylor's Blue and Toluidine blue are important dyes for the induction of metachromasia, which is the ability of dyes to color different tissue constituents in different color (Moura et al., Curr. Drug. Targ., 4, 133-141 (2003). Toluidine blue is one of the more popular dyes used for staining microorganisms and can also be used for the diagnosis of several diseases (Moulder et al., Textbook of Microbiology, 19th Ed., Saunder, Philadelphia, pp. 18 and 47, )1968)).

Methylene blue has been approved for oral administration and has been reported to be effective as an antiseptic, disinfectant, and antidote for cyanide and nitrate poisoning. Methylene blue, injected intravenously at a dose of 1 mg/kg body weight, is effective in the treatment of methemoglobinemia, a clinical disorder where more than 1% of the hemoglobin in the blood has been oxidized to Fe³⁻. Drug Facts and Comparisons, page 1655 (J. B. Lippincott Co., St. Louis, Mo. 1989) reports that methylene blue is useful as a mild genitourinary antiseptic for cystitis and urethritis, in the treatment of idiopathic and drug-induced methemoglobemia and as an antidote for cyanide poisoning. Recommended dosages are 55 to 130 mg three times daily, administered orally. Oral absorption is 55% to 97%, averaging 74%, DiSanto and Wagner, J. Pharm. Sci. 61(7), 1086-1090 (1972). Pharmacopeia states that the recommended dose is 50 to 300 mg by mouth and 1 to 4 mg/kg body weight intravenously. Side effects include blue urine, occasional nausea, anemia and fever. American Hospital Formulary Service “Drug Information 88” states that the recommended intravenous dosage for children is 1 to 2 mg/kg body weight, injected slowly over several minutes, which can be repeated after an hour. 55 mg tablets are available from Kenneth Manne. 65 mg tablets are available from Star Pharmaceuticals. Methylene Blue Injection (10 mg/ml) is available from American Reagent, Harvey, Kissimmee, Pasadena.

Narsapur and Naylor reported in J. Affective Disorders 5, 155-161 (1983) that administrative of methylene blue orally, at a dosage of 100 mg b.i.d. or t.i.d., or intravenously, 100 mg infused over 10 min, may be effective in treating some types of mental disorders in humans, indicating that the dye may cross the blood-brain barrier and therefore have particular applicability in the treatment of viral infections of the brain and central nervous system. Methylene blue was administered for periods of one week to 19 months to adult humans, with minimal side effects.

The American Hospital Formulary Service “Drug Information 88” reports that methylene blue is absorbed well from the GI tract, with about 75% excreted in urine and via the bile, mostly as stabilized colorless leukomethylene blue. As reported by G. E. Burrors in J. Vet. Pharmacol. Therap. 7, 225-231 (1984), the overall elimination rate constant of methylene blue, in sheep, is 0.0076±0.0016 min⁻¹, with minimal methemoglobin production at doses as high as 50 mg/kg and no hematologic changes seen up to four weeks after a total dose of 30 mg/kg methylene blue. The 24 h LD₅₀ for intravenous methylene blue administered as a 3% solution was 42.3 mg/kg with 95% confidence interval limits of 37.3 to 47.9 mg/kg, demonstrating that methylene blue can be safely administered at a dosage of up to at least 15 mg/kg. As reported by Ziv and Heavner in J. Vet. Pharmacol. Therap. 7, 55-59 (1984), methylene blue crosses the blood-milk barrier easily.

U.S. Pat. No. 6,346,529 to Floyd, et al. describes the use of methylene blue and other thiazine dyes to inactivate HIV. It also demonstrates that the effect of the dye on different types of viruses is unpredictable, and that one cannot use results with one virus to predict efficacy with another. See Table 4, comparing efficacy against HIV with a lack of efficacy against Herpes Simplex Virus type 1 and type 2.

In contract, U.S. Pat. No. 5,545,516 to Wagner describes the inactivation of extracellular enveloped viruses in blood and blood components by phenthiazin-5-ium dyes plus light. The described process inactivates pathogenic contaminants in whole blood, plasma, cellular blood components, by adding a phenthiazin-5-ium dye(s) thereto and irradiating the dye-containing composition with light of wavelengths from 560 to 800 nm or red light, such that they are suitable for transfusion. Obviously the conditions for treating blood products in a laboratory, and the availability of a radiant light source are quite different from the conditions required to treat a patient with a chronic viral conditions such as hepatitis C

The compounds described herein have the chemical formula shown below: 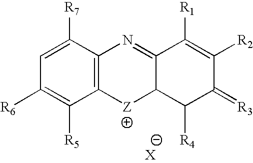
wherein R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen; R₃ and R₆ are independently selected form the group consisting of —O, —NH₂, —NHR₈, and —NR₉R₁₀ wherein R₈—R₁₀ is a linear, branched or cyclic hydrocarbon or R₉ and R₁₀ together with the nitrogen atom to which they are attached form an optionally substituted 5-, 6-, or 7-membered ring, optionally containing one or more heteroatoms selected from non-peroxide oxy (—O—), thio (—S—), sulfinyl (—SO—), sulfonyl (—So₂—), or amine (—NR—), where R is hydrogen; linear, branched, or cyclic hydrocarbon; linear, branched or cyclic substituted hydrocarbon aryl, or substituted aryl; X⁻ is a counter ion and Z is either S or O.

Examples of useful thiazine dyes include, but are not limited to, methylene blue, methyl methylene blue, dimethyl methylene blue, azure A, azure B, azure C, methylene green, new methylene blue, Taylor's Blue, Toluidine Blue O, and thionine. These dyes are all commercially available from a number of different sources. Symmetrical 3,7bis(dialkylamino)phenothiazin-5ium derivatives which may be useful are described in Moura et al., Current Drug Targets, Vol. 4, 133-141 (2003). Derivatives of methylene blue in which the methyl groups of methylene blue have been replaced with ethyl, n-propyl, n-butyl, n-pentyl, and n-hexyl groups are described in Mellish et al., Photochemistry and Photobiology, Vol. 75, No. 4, pp. 392-397 (2002). Finally, phenoxazine dyes, in which the sulfur atom of the thiazine ring is replaced by an oxygen atom, may also be used. Examples of phenoxazine dyes include Nile Blue and its derivatives.

Methylene blue and its derivatives typically exist as the chloride or bromide salts; however, other anions can be used to stabilize the positive charge on the molecule. Suitable anions include inorganic anions such sulfate, sulfamte, phosphate, nitrate, and nitrite; and organic anions such as acetate, propionate, succinate, glycolate, lactate, malate, tartarate, citrate, ascorbate, pamoate, maleate, hydroxymaleate, phenylacetate, glutamate, benzoate, salicylate, sulfanilate, 2- acetoxybenzoate, fumarate, tolunesulfonate, naphthalenesulfonate, methanesulfonate, ethane disulfonate, oxalate, and isethionate salts.

Combinations with Radiation or Other Active Compounds

The activity of the dye can be enhanced further by irradiation with light or by derivatization with compounds such as antisense mRNA.

The thiazine dye can also be provided in combination with other therapeutic, prophylactic or diagnostic agents.

For example, methylene blue or a derivative of methylene blue can be administered adjunctively with other active compounds such as analgesics, anti-inflammatory drugs, antipyretics, antidepressants, antiepileptics, antihistamines, antimigraine drugs, antimuscarinics, anxioltycis, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. Suitable anti-inflammatory agents include, but are not limited to, cortisone, hydrocortisone, betamethasone, dexamethasone, fluocortolone, prednisolone, triamcinolone, indomethacin, sulindac.

Alternatively, the formulation can include other antiinfectives such as antibiotics, antifungals, other antivirals, amoebicidal, trichomonocidal, to treat viral or secondary infections. Suitable antibiotics include, but are not limited to, beta-lactam antibiotics, chloramphenicol, rifampin, clarithromycin, adriamycin, erythropoietin, neomycin, gramicidin, bacitracin, sulfonamides, and nalidixic acid. Suitable anti-fungals include, but are not limited to, voriconazole (Vfend®), azoles, imidazoles, polyenes, posaconazole, fluconazole, intraconazole, amphotericin B, 5-flurocytosine, miconazole, and ketoconazole. Suitable antivirals include, but are not limited to, acyclovir, amantadine, rimantadine, nevirapine, cidofovir (Vistide™), trisodium phosphonoformate (Foscarnet™), famcyclovir, pencyclovir, valacyclovir, zidovudine (AZT, Retrovir™), didanosine (dideoxyinosine, ddl, Videx™), stavudine (d4T, Zerit™), zalcitabine (dideoxycytosine, ddC, Hivid™, nevirapine (Viramune™, lamivudine (Epivir™. 3TC), saquinavir (Invirase™). Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), interferon, cyclovir, alpha-interferon, ribavirin, and interferon or combinations of ribavirin and interferon or beta globulin.

C. Additives, Excipients and Carriers

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, and coating compositions.

Carrier also includes all components of the coating composition which may include plasticizers, pigments, colorants, stabilizing agents, and glidants. The delayed release dosage formulations may be prepared as described in standard references such as “Pharmaceutical dosage form tablets”, eds. Liberman et. al. (New York, Marcel Dekker, Inc., 1989), “Remington—The sicence and practice of pharmacy”, 20th ed., Lipincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formulation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and metharcrylic acid copolymers, metharcrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, peg-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta-.alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic axillary substances such as wetting or emulsifying agents, dyes, pH buffering agents, or preservatives.

The compounds can be administered as tablets, hard or soft shell capsules, suspensions or solutions. Devices with different drug release mechanisms can be combined in a final dosage form comprising single or multiple units. Examples of multiple units include multilayer tablets, capsules containing tablets, beads, granules, etc. For example, an immediate release portion can be added to the extended release system by means of either applying an immediate release layer on top of the extended release system by means of either applying compression process or in a multiple unit system such as a capsule containing extended and immediate release beads.

### Extended Release Formulations

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol® 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical table binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking to the die. The lubricant is chosen from such slippery solids as talc, magnesium and calcium stearate, stearic acid and hydrogenated vegetable oils.

Extended release tablets containing wax material are generally prepared using methods known in the art such as a direct blend method, a congealing method, and an aqueous dispersion method. In a congealing method, the drug is mixed with a wax material and either spray congealed or congealed and screened and processed.

### Delayed Release Formulations

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coating using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt % to 50 wt % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

II. Methods of Manufacturing

As will be appreciated by those skilled in the art and as described in the pertinent tests and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1005).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Alternatively, the dye can be continuously delivered to a patient over an extended period of time using a controlled release polymeric implant or implantable pump. Polymeric implants are generally manufactured from polymers which degrade in vivo over a known period of time. Examples of useful polymers include polyanhydrides, polylactic acid, polyorthoester, and ethylene vinyl acetate. These devices are also commercially available. Alza Corporation, Palo Alta, Calif., and Nova Pharmaceuticals, Baltimore, Md., both manufacture and distribute biodegradable controlled release polymeric devices.

The thiazine dyes can also be delivered using techniques known to those skilled in the art of drug delivery to target specific cell types or to enhance the activity of the dye. For example, a procedure utilizing injection of photoactive drugs for cancer treatment is described by Edelson, et al., in New England J. Med. 316, 297-303 (1987). Thiazine dyes can be specifically delivered to macrophages, a site of high hepatitis virus concentration in hepatitis virus patients, using techniques such as liposome delivery. Liposomes are generally described by Gregoriadis, Drug Carriers in Biology and Medicine Ch. 14, 287-341 (Academic Press, N.Y., 1979). Methods for making light sensitive liposomes are described by Pidgeon, et al., in Photochem. Photobiol. 37, 491-494 (1983). Liposome compositions are commercially available from companies such as the Liposome Company, Inc., Princeton, N.J. Release of compounds from liposomes ingested by macrophages is described by Storm, et al., in Biochim. Biophys. Acta 965, 136-145 (1988).

III. Methods of Treatment

A. Viral Diseases to be Treated

### Hepatitis

Hepatitis is inflammation of the liver. Several different viruses cause viral hepatitis. They are named the hepatitis A, B, C, D, and E viruses. Currently seven viruses, A, B, C, D, E, G and transfusion transmitted virus (TTV) are recognized in the hepatitis virus alphabet. Hepatitis G virus and TTV probably do not cause liver disease in humans. Hepatitis A and E usually cause a self-limiting hepatitis followed by complete recovery but occasionally cause fulminant hepatic failure. Hepatitis B and C are major public health problems worldwide due to their sequelae of chronic hepatitis, cirrhosis and primary liver cancer. Chronic hepatitis C is a particular health issue for Western Europe already, accounting for 40% of end-stage cirrhosis and 30% of liver transplants. The contribution of hepatitis C to chronic liver disease is predicted to rise in the future. Vaccines can prevent hepatitis A and B. Interferon alpha is effective treatment in 25-30% of patients with chronic hepatitis B or C. The prospects for treating chronic hepatitis B have been improved by the introduction of reverse transcriptase inhibitors. Lamivudine is the first drug of this class to be licensed. The optimal use of these new drugs is currently being studied. The success rate for treating chronic hepatitis C can be raised to about 40% with combination therapy of interferon alpha and ribavirin. All of these viruses cause acute, or short-term, viral hepatitis. The hepatitis B, C, and D viruses can also cause chronic hepatitis, in which the infection is prolonged, sometimes lifelong. Other viruses may also cause hepatitis, but they have yet to be discovered and they are obviously rare causes of the disease.

Symptoms of Viral Hepatitis include jaundice (yellowing of the skin and eyes), fatigue, abdominal pain, loss of appetite, nausea, vomiting, diarrhea, low grade fever, and headache.

Hepatitis A is primarily spread through food or water contaminated by feces from an infected person. Rarely, it spreads through contact with infected blood. Hepatitis A is prevented by a vaccine; by avoiding tap water when traveling internationally and practicing good hygiene and sanitation. Hepatitis A usually resolves on its own over several weeks. Hepatitis B is spread through contact with infected blood, through sex with an infected person, and from mother to child during childbirth. Hepatitis B can be prevented by a vaccine and treated with alpha interferon, peginterferon, lamivudine, or adefovir dipivoxil. Acute hepatitis B usually resolves on its own. Very severe cases can be treated with lamivudine.

Hepatitis C is a viral infection of the liver. The virus, HCV, is a major cause of acute hepatitis and chronic liver disease, including cirrhosis and liver cancer. HCV is spread primarily by direct contact with human blood. The major causes of HCV infection worldwide are use of unscreened blood transfusions, and re-use of needles and syringes that have not been adequately sterilized. There is no vaccine for hepatitis C; the only way to prevent the disease is to reduce the risk of exposure to the virus. Current means for treating chronic hepatitis C is with peginterferon alone or in combination treatment with peg-interferon and the drug ribavirin. Acute hepatitis C: treatment is recommended if it does not resolve within 2 to 3 months.

Hepatitis D is also spread through contact with infected blood. This disease occurs only in people who are already infected with hepatitis B. Treatment of chronic hepatitis D is with alpha interferon. Hepatitis E is spread through food or water contaminated by feces from an infected person. There is no vaccine for hepatitis E; the only way to prevent the disease is to reduce the risk of exposure to the virus. Hepatitis E usually resolves on its own over several weeks to months.

Some cases of viral hepatitis cannot be attributed to the hepatitis A, B, C, D, or E viruses. This is called non A-E hepatitis.

Chronic infection with the hepatitis C virus (HCV) is common and affects up to 1% of the UK population. It is well recognized that chronic HCV infection is associated with a wide variety of symptoms including fatigue, upper abdominal pain and dyspepsia leading to an overall reduction in quality of life. It has been shown that effective elimination of the hepatitis C virus with interferon and ribavirin leads to an improvement in symptoms but therapy with these agents if effective in only a small proportion of patients (40%). Hence there is a pressing need for effective therapies that improve the quality of life of patients with chronic hepatitis C.

The World Health Organization has declared hepatitis C a global health problem, with approximately 3% of the world's population (roughly 170-200 million people) infected with HCV. In the U.S. approximately 3 million people are chronically infected, many of whom are still undiagnosed. According to the Sustainable Sciences Institute (SSI) the situation is quire worse in Egypt. Egypt has a population of 62 million and contains the highest prevalence of hepatitis C in the world. The national prevalence rate of HCV antibody positivity was estimated by the Egyptian Ministry of Health (MOHP) in 1999 to be 18.9%. Since 25-30% of individuals clear the infection, the estimated adjusted national prevalence rate is 12% (or 7.2 million people) (MOHP, 1999).

Hepatitis C virus was first described in 1989 as the putative viral agent of non-A non-B hepatitis. It is a member of the Flaviviridae family and has been recognized as the major causative agent of chronic liver disease, including chronic active hepatitis, cirrhosis and hepatocellular carcinoma. HCV is a positive RNA virus with a genome containing approximately 9500 nucleotides. It has an open reading frame that encodes a large polyprotein of about 3000 amino acids and is characterized by extensive genetic diversity.

HCV has six major genotypes (subtypes): 1a, 1b, 2a, 3, 4, 5, and 6. Gentotypes 1a and 1b, which are the most common in the U.S., are more difficult to treat. Interestingly, genotype 4 represents over 90% of cases in Egypt. Chronic HCV is the main cause of liver cirrhosis and liver cancer in Egypt, and indeed, one of the top five leading causes of death. In Egypt, the major route of exposure appears to be due to medical therapy and inadequate sterilization techniques and supplies. In addition to blood transfusions prior to 1994, the major risk factor associated with HCV infection is a history of anti-schistosomal injection treatment. Schistosomiasis is a common parasitic disease in Egypt acquired through swimming or wading in contaminated irrigation channels or standing water. Thus, farmers and rural populations are at greatest risk, and this is supported by the higher prevalence rate of HCV in the Nile delta and rural areas. Schistosomiasis can lead to urinary or liver damage over many years. Prior to 1984 the mainstay of treatment was intravenous tartar emetic. Widespread treatment campaigns were carried out in the countryside of Egypt in the 70's and early 80's. Needles were routinely recycled and not properly sterilized at that time due to cost and limited resources. Overall, despite improvement in schistosomiasis-induced morbidity this campaign set the stage for the current large hepatitis disease burden in Egypt. Further, with such a high prevalence rate, transmission of hepatitis C through unusual routes has become significant. For example, tattooing, circumcision or other medical procedures performed by non-medical personnel are more common routes of infection in Egypt than elsewhere. In addition, household transmission, vertical and sexual transmission routes are also under investigation.

As expected, the availability and cost of treatment for hepatitis C in Egypt is prohibitive. Although the most common methods of previous hepatitis C transmission (injection-based treatment for schistosomiasis and blood transfusions) have been addressed, the prevalence in those under age 20 is still approximately 10%, demonstrating the continued presence of significant hepatitis C transmission in modern-day Egypt.

### Latent Viruses

Clinically important DNA-viral infections are caused by viruses such as Herpes simplex virus type 1 & 2, Varicella zoster virus, Epstein-Barr virus, Cytomegalovirus, and Herpes virus type 6 & 7, Adenovirus, and Human polyoma viruses, e.g. JC virus and BK virus.

The papovaviruses (JC and BK) are widely distributed in the human population, as evidenced by the presence of specific antibodies in 70-80% of adult sera. BK virus has been associated with hemorrhagic cystitis and is capable of inducing obstructive renal failure. The JC virus is thought to persist in the kidney, and is reactivated when the host immune system is impaired (e.g., HIV infection, immunosuppressive therapy, pregnancy). In addition, progressive multifocal leukoencephalopathy (PML) caused by JC virus may occur when patient receives profound immunosuppressive therapy, such as chemotherapy or radiation.

Adenoviruses (AdV) cause acute disease of the respiratory and gastrointestinal tracts. The high incidence of adenovirus infections in organ transplant (kidney, bone marrow) recipients and AIDS patients suggests that these infections most probably represent reactivation of a latent adenovirus infection. Pneumonia and hepatitis are the most common infections. Hemorrhagic cystitis occasionally occurs and has been associated with specific serotypes of the virus. Infections in transplant patients caused by adenovirus occurs in approximately 17% of patients who receive allogeneic bone marrow transplantation. Reactivation infections by B or C (AdV2, AdV5) serotypes are most common. No specific treatment is available.

Herpes viruses are a leading cause of human viral disease, second only to influenza and cold viruses. Infection is life long and the viruses are capable of causing overt disease or remaining silent for many years only to be reactivated, for example as shingles. There are 25 families in the Herpetoviridae but only six of them are know to infect man with any regularity, Herpes simplex virus Type 1 (HSV-1), Herpes simplex virus Type 2 (HSV-2), Epstein Barr virus (EBV), Cytomegalovirus (CMV), Varicella Zoster Virus (VZV), Human herpes virus 6 (exanthum subitum or roseola infantum), and Human herpes virus 8 (Kaposi's sarcoma-associate herpes virus).

There are two types of Herpes simplex virus (HSV), HSV-1 and HSV-2. Herpes simplex 1 and 2 can infect both humans and animals but only humans show symptoms of disease HSV-1 and HSV-2 first infect cells of the mucoepithelia or enter through wounds. They then frequently set up latent infections in neuronal cells. Both types of HSV can also persistently infect macrophages and lymphocytes.

A large proportion of the population has evidence of HSV-1 infection. In underdeveloped countries, HSV-1 antibodies are found in more than 90% of children as a result of poor hygiene. HSV-2 is normally spread sexually. HSV-2 infections are more prevalent later in life as the number of sexual contacts increases. Thus, the lowest rates of infection are found in children and the highest rates in prostitutes among whom as many as 80% are infected with HSV-2.

Herpes simplex 1 and 2 are frequently benign but can also cause severe disease. Diseases caused by HSV-1 and/or HSV-2 include, oral herpes herpes pharyngitis, herpes keratitis, which is a leading cause of corneal blindness in the United States, herpes whitlow, herpes gladiatorum eczema herpeticum, genital herpes, HSV encephalitis, and HSV meningitis.

HSV reactivation of infection often occurs at the same site as the initial infection. There are several agents that seem to trigger reactivation, most of which are stress-related. It also appears that exposure to strong sunlight and perhaps fever can lead to reactivation. These factors may cause some degree of immune suppression that leads to renewal of virus proliferation in the nerve cell. HSV types 1 and 2 are a common cause of lesions impatient with malignancy. HSV infection results in most cases from reactivation of latent virus. More than 80% of bone marrow transplant (BMT) patients have reactivation of latent HSV residing in the neuronal ganglia. The same is true for patients with leukemia, lymphoma, or solid tumors who receive intensive chemotherapy. This reactivation usually occurs within the first 3 weeks after transplantation or chemotherapy and is characterized by ulcerative oral lesions (in 85% of the cases) or genital lesions (in 15% of the cases). Other possible presentations of HSV infection in immunocompromised patients are esophagitis, tracheitis, pneumonitis, and, rarely, encephalitis. When pneumonitis occurs, it may present as a local infiltrate (usually originating from aspiration of the virus from the upper airways) or as a diffuse interstitial infiltrate (from viremia).

Varicella-Zoster Virus (also known as Herpes Zoser Virus, Human Herpes Virus3) causes two major disease, chickens-pox (Varicella), usually in childhood, and shingles, later in life. Shingles (Zoster) is a reactivation of an earlier varicella infection.

The Varicella-Zoster virus may be reactivated under stress or with immune suppressing. VZV infection in cancer patients can present either as primary infection (chickenpox) or as reactivation (shingles). Children with acute leukaemia who develop varicella are at particular high risk for VZV pneumonia which may occur in up to one-third of patients with a fatality rate of about 10% (Feldman, S. and Lott, L., Pediatrics 80:465-472 (1987)). Patients with AIDS often exhibit reactivation of varicella infection. Increased reactivation of varicella-zoster virus is also observed in CD34+ allogenic stem cell transplantation (Martino, R., et al., Haematologica. 86(10):1075-86 (2001)). Herpes zoster is most frequently observed among cancer patients with leukaemia or lymphoma and in recipients of autologous or allogeneic stem cell transplants (SCT). For chickenpox or zoster in immunodeficient cancer patients, intravenous acyclovir is used for treatment. For treatment of localised zoster among patients with mild to moderate immunosuppression, high-dose oral acyclovir, valaciclovir or famciclovir are often used. However, these drugs have significant drawbacks as discussed above. Following allogeneic SCT, VZV reactivation may occur for a prolonged period of time. However, long-term antiviral drug prophylaxis is not advisable in allograft recipients, since it only delays the occurrence of zoster and carries the potential for induction of VZV resistance. Epstein-Barr Virus (EBV) occurs worldwide. In the United States, as many as 95% of adults between 35 and 40 years of age have been infected. When infection with EBV occurs during adolescence or young adulthood, it causes infectious mononucleosis 35% to 50% of the time. Although symptoms of infectious mononucleosis usually resolve in one or two months, EBV remains dormant or latent in a few cells in the throat and blood for the rest of the person's life. Periodically, the virus can reactivate. In a few carriers of EBV the emergence of Burkitt's lymphoma and nasopharyngeal carcinoma, two rate cancers is seen. The tumor cells show evidence of EBV DNA and tumor antigens and patients show a much higher level of anti-EBV antibodies than other members of the population. Further evidence that implicates EBV in Burkitt's lymphoma is the observation that EBV can transform B lymphocytes in culture and can produce B cell lymphomas in primates.

If cell mediated immunity is suppressed, resolution of the EBV disease may not occur. Uncontrolled viral replication may lead to a severe syndrome with B cell lymphoproliferation, leukopenia and lymphoma. In patients with T cell deficiency X-linked lymphoproliferative disorder may occur. Transplant patients and AIDS patients who are also immunosuppressed may exhibit post-transplant lymphoproliferative disorder. In one study, reactivation of the Epstein-Barr virus (EBV) after allogeneic stem cell transplantation (allo-SCT) was observed in 31% of patients (Van Esser, et al., Blood 98(4):972-978 (2001).

Unlike herpes simplex virus, there are no drugs available to treat Epstein-Barr virus. This may reflect the absence of a thymidine kinase encoded by this virus (drugs such as acyclovir that are active against herpes simplex are activated by the viral thymidine kinase).

Cytomegalovirus (CMV) infection is found in a significant proportion of the population. By college age, about 15% of the US population is infected and this rises to about half by 35 years of age. The virus is spread in most secretions, particularly saliva, urine, vaginal secretions and semen. Cytomegalovirus infection is therefore sexually transmitted. It can also spread to a fetus in a pregnant woman and to the newborn via lactation. In the hospital, the virus can also be spread via blood transfusions and transplants. In third world countries with more crowded conditions, the virus is found in a much higher proportion of the population than in western countries.

Cytomegalovirus causes no symptoms in children and at most mild disease in adults. However, CMV infections can decrease blood cell count, resulting in dangerous neutropenia or slight but persistent thrombocytopenia. It can also induce organ specific diseases, like CMV hepatitis, pneumonitis, or GI infections. Of these, the most deadly infection is known to be CMV pneumonitis. CMV pneumonitis occurs in 15% of patients with CMV reactivation. CMV is the most common viral cause of congenital disease. Up to one in forty newborns in the United States are infected by the virus. Abnormalities include microcephaly, rash, brain calcification and hepatosplenomegaly. These may result in hearing loss (bilateral or unilateral) and retardation.

Although CMV is suppressed, the virus may later reactivate, particularly in cases of immunosuppression; indeed, infection by the virus can, itself, be immunosuppressive. Mild forms of the illness may manifest as fever, hepatitis, leukopenia, and thrombocytopenia. Severe forms manifest as interstitial pneumonia (with a mortality rate of 80% to 90%) and gastroenteritis. Patients receiving cytoreductive therapy for acute leukaemia and recipients of allogeneic SCT are at high risk for serious CMV disease following reactivation of virus. Among adults with acute leukaemia, CMV pneumonia was reported to occur in 2.9% of patients with a case-fatality rate of 57% (Nguyen Q. et al., Clin Infect Dis 32:539-45(2001)). In patients who have received an organ transplant or have an immunosuppressive disease (e.g. AIDS), Cytomegalovirus can be a major problem. Reactivation of CMV occurs in approximately 80% of patients who are seropositive before transplantation. Most commonly, infection appears 4 to 10 weeks after transplantation. CMV can cause multiorgan disease after stem cell transplant (SCT), including pneumonia, gastroenteritis, hepatitis, retinitis, and encephalitis. In certain allografts, T-cells and or B-cells are removed from the graft to prevent chronic graft-versus-host disease (GVHD), studies have shown that transplantation of T-cell depleted grafts is associated with more frequent viral reactivation, longer time to CMV clearance with antiviral therapy, and a slight increase in the incidence of CMV disease (Hebart, H., Blood 97(7):2183-2185 (2001).

Prophylactic high-dose intravenous acyclovir mediates only partial protection from CVM disease after allogeneic SCT, and is ineffective in autograft recipients (Boeckh M, et al., J Infect Dis 172:939-43 (1995)). Intravenous ganciclovir prophylaxis results in less frequent CMV disease but not in improved survival.

Human herpes virus 6 is found worldwide and is found in the saliva of the majority of adults (>90%). It infects almost all children by the age of two. It can set up a latent infection in T cells which can later be reactivated when cells are stimulated to divide. Human herpes virus-6 has two forms, HHV-6A and HHV-6B. The latter causes exanthem subitum, otherwise known as roseola infantum. Symptoms include fever and sometimes upper respiratory tract infection and lymphadenopathy. In adults, primary infection is associated with a mononucleosis. This virus may co-infect HIV-infected T4 lymphocytes exacerbating the replication of HIV. Patients with HIV have a higher infection rate than the normal population. HHV-6 has been associated with a number of neurological disorders, including encephalitis and seizures. It has been postulated to play a role in multiple sclerosis and chronic fatigue immunodeficiency syndrome. Cell-mediated immunity is essential in control, although infection is life-long and the virus can reactive in patients who are immunosuppressed.

Human herpes virus 7 is found in the saliva of the majority of the adult population (>75%). Most people acquire the infection as children and it remains with them for the rest of their lives. It is similar to HHV-6 and may be responsible for some cases of exanthem subitum.

Human herpes virus 8 formerly known as Kaposi's sarcoma associated herpes virus is found in the saliva of many AIDS patients.

There are a variety of nucleoside analog drugs used to treat herpes infections, many of which are of high specificity since they take advantage of the activation of the drug by a viral enzyme, thymidine kinase. The best known of the nucleoside analogs is acycloguanosine (acyclovir) but there are other approved drugs including famciclovir and valacyclovir. All of these nucleoside analogs suffer from the appearance of resistant herpes mutants. It should be noted that these drugs act against the replicating virus (they are incorporated into the DNA as it is copied) and therefore they are ineffective against latent virus.

The emergence of resistant herpes virus strains that cause disease unresponsive to antiviral drugs is reported with increasing frequency in immunocompromised hosts (Englund J A, et al., Ann Intern Med 112:416-22 (1990); Reusser P., J Hosp Infect 33:235-48 (1996); Jabs D A, Enger C, et al., J Infect Dis 177:770-3 (1998); Chakrabarti S, J Infect Dis 181:2055-8 (2000); Boivin G, J Infect Dis 184:1598-602 (2001)). Acyclovir-resistant HSV disease developed in 7 to 14% of SCT patients who are given acyclovir treatment (Englund J A, et al., Ann Intern Med 112:416-22 (1990)). CMV resistance to ganciclovir is observed in up to 27% of patient with AIDS receiving longterm ganciclovir therapy (Reusser P., J Hosp Infect 33:235-48 (1996)). The presence of multidrug-resistant HSV or CMV strains was documented in several SCT recipients, and resistance to foscarnet was not infrequent (Chakrabarti S, J Infect Dis 181:2005-8 (2000)).

Herpes B is a simian virus found in old world monkeys such as macaques but it can be a human pathogen in people who handle monkeys (monkey bites are the route of transmission). In humans, the disease is much more problematic than it is in its natural host. Indeed, about 75% of human cases result in death with serious neurological problems (encephalitis) in many survivors. There is also evidence that the disease can be passed from a monkey-infected human to another human. In vitro the virus is sensitive to both Acyclovir and Ganciclovir and these are recommended for therapy. Their efficacy is unknown.

Hepatitis B is spread through contact with infected blood, through sex with an infected person, and from mother to child during childbirth. Hepatitis B can be prevented by a vaccine and treated with alpha interferon, peginterferon, lamivudine, or adefovir dipivoxil. Acute hepatitis B usually resolves on its own. Very severe cases can be treated with lamivudine.

Reactivation of hepatitis B has been described under a variety of circumstances. Reactivation can occur spontaneously (Davis G L., et al., Gastroenterology 86:230-235 (1984) and Fattovich G, et al., Liver 10:141-146 (1990)); under impaired immune status, e.g. presence of malignancies or after chemotherapy (Hoofnagle J H, et al., Ann. Intern. Med. 96:447-449 (1982) and Martin B A, et al., Bone Marrow Transplant 15:145-148 (1995)) and in patients with AIDS (Vento S, et al., Lancet i:332-333 (1989)); following transplantation, e.g. bone marrow (Martin B A , et al., Bone Marrow Transplant 15:145-148 (1995)) and renal transplantation (Marcellin P, et al., Gastroenterology 100:1432-1434 (1991); following resection for hepatocellular carcinoma (Kubo S, et al., Ann Surg 223: 139-145 (2001)); chemotherapy treatment (Carman W F, Lancet 345:1406-1407 (1995)); following withdrawal of immunomodulatory drugs such as steroids, methotrexate, cyclosporine and chloroquine (Rostoker G. et al., Nephron 56:224-224 (1990); Flowers M A, et al., Ann. Intern Med. 112:381-382 (1990); Gruber A, et al., J. Intern. Med. 234:223-225 (1993); Helbling B and Reichen J. Schweiz. Med. Wochenschr. 124:759-762 (1994)); and in pregnancy (Rawal B K, et al., Lancet 337:364-364 (1991)).

Patients with lymphoma are more prone to reactivation than patients with other malignancies (Yeo W, et al., J Med Virol 62:299-307 (2000)). For cancer patients receiving cytotoxic chemotherapy, hepatitis B virus (HBV) reactivation is a well described complication resulting in varying degrees of liver damage. In one study, over 60% of patients undergoing cytotoxic therapy showed reactivation (Los A S, et al., Gastroenterology 100:182-188 (1991)). In another study, 138 consecutive cancer patients who were HBV carriers and undergoing chemotherapy were studies, 36 (26%) developed HBV reactivation (Yeo, W., et al., British Journal of Cancer 90, 1306-1311(2004)). It has been estimated that in several developing countries, the carriage of hepatitis B virus (HBV) in cancer patients may be as high as 12%, and such patients are at risk of developing fatal HBV reactivation during chemotherapy (Yeo, W., et al., J Med Virol. 70(4):553-61 (2003)). For patients with breast cancer undergoing chemotherapy treatment, it was demonstrated that 41% developed HBV reactivation, which resulted in premature termination of chemotherapy treatment in 71% of these patients (Yeo, W., et al., J Med Virol. 70(4):553-61 (2003)). Treatment of reactivation usually follows the usual recommendations. The use of lamivudine before commencing chemotherapy in HBsAg-positive cancer patients reduces the incidence of HBV reactivation. However, improvement in survival was not observed (Yeo, W., et al., Journal of Clinical Oncology 22(5):927-934 (2004)).

Methods of Administration and Effective Dosages

The drug is preferably administered orally, although it can also be administered by injection. The preferred dosage range for methylene blue is 30 to 180 mg twice a day, more preferably between 60 and 130 mg twice a day, for an immediate release formulation. An equivalent dosage can be administered in a controlled release formulation. In some cases it may be desirable to administer the formulation using a dosage escalation regime to reach a desired maintenance level. The appropriate in vivo dosage can be determined by extrapolation from in vitro levels, assuming the usual blood volume for adult humans if approximately 10, and taking into account the 74% oral absorption and 75% excretion of that absorbed over a period of time, and assuming the lower therapeutic index in darkness than in light.

The formulation can also be administered as spray dried particles, for intranasal or pulmonary administration, with or without a surfactant or other carrier.

The method described herein does not require administration of exogenous light, although the results may be enhanced by exposure to light in addition to that normally transmitted through the skin. Exposure to light can occur with exposure to sun light, a tanning light, or even incandescent light.

In the case of treating patients with latent viral infection or who will or are immunosuppressed or undergoing chemotherapy, such as cancer patients and transplant recipients, the drug can be administered prior to or following reactivation of the virus. The drug can be administered for an effective period of time to clear the viral infection, which can be from days to months to years. Suitable lengths of treatment for viral reactivation infections include, but are not limited to, 4 weeks (about 1 month), 12 weeks (about 3 months), 24 weeks (about 6 months), 48 weeks (about 1 year), or even longer as necessary. In a preferred embodiment, patients are treated from 3 to 6 months. In a more preferred embodiment, patients are treated for a least 50 days (about two months) up to 48 weeks. Longer treatment times, on the order of about 48 weeks, or even longer, may be necessary for patients who have responded poorly to other anti-viral treatments, relapse patients, patients with more than one viral infection and patients with complications due to viral infections. For the treatment of hepatitis, the compositions should be administered for an effective period of time, for example, for at least one month.

Combination Therapy with Radiation or other Drugs

The method described herein does not require administration of exogenous light, although the results may be enhanced by exposure to light in addition to that normally transmitted through the skin. Exposure to light can occur with exposure to sun light, a tanning light, or even incandescent light.

Combination therapy can be sequential, meaning treatment with one agent first followed by treatment with a second agent, or it can be simultaneous, meaning treatment with both agents at the same time. If the combination therapy is sequential, administration of a second agent occurs within a reasonable time after administration of the first agent. If the combination therapy is simultaneous, both agents can be administered at the same time in the same dosage form or in separate dosage forms. The exact regimen will depend on the severity of the disorder and the response to the treatment.

All publications cited are incorporated by reference.

The present invention will be further understood with reference to the following examples.

### EXAMPLES

### EXAMPLE 1

Clinical Trial to Demonstrate Efficacy in Reduction in Hepatitis C Viral Load by Administration of Oral Methylene Blue.

This was an open label study of methylene blue assessing its effectiveness in reducing serum viral load and treating the symptoms associated with chronic hepatitis C.

Clinical Study:

The study was to evaluate the effectiveness of Methylene Blue in reducing the viral titer and alleviating the related symptoms of chronic Hepatitis C infection. The study design was an open label study to assess the reduction of viral titres in patients over a 24 week treatment period and subsequent 4 week follow-up period.

The dose was 65 mg of Methylene Blue twice daily in the capsules.

They type of subjects were male and female subjects aged between 18 and 70 with proven chronic hepatitis C, 60% with and 40% without cirrhosis, who are refractory to, unwilling, or unable to take interferon/ribavarin therapy. A total of 60 subjects were enrolled to ensure that 40 completed the treatment period.

25 patients with hepatitis C, genotype 4 received Methylene Blue 6mg b.d for 100 days. The viral load was measured by PCR at days 50 and 100.

The primary endpoint was change in Hepatitis C viral titers during treatment and after the treatment period.

The secondary endpoints were to reduce hepatic inflammation (measured as an improvement in liver function tests) in patients with chronic hepatitis C.

The primary efficacy parameters were (1) Overall reduction in hepatitis C viral titer after the period of treatment. (2) Early Viral Response (EVR) rate as defined as ≧2 log reduction in viral count after 90 days of treatment. (3) the Sustained Viral Reduction (SVR) after 6 months of treatment in patients who obtained an EVR.

Secondary objectives included:
to indicate whether patients on active treatment show significant reduction in ALT when compared to the placebo group
to indicate whether patients on active treatment show significant reduction in AST when compared to the placebo group
to demonstrate the clinical safety of the product.
To show an improvement in FSS.

Study medication was taken over a 24-week period, and patients attended a follow-up visit at four weeks after the final dose was taken.

Subjects met all of the following criteria at screening in order to be considered for this study.
Male or female aged 18-70 inclusive on date of screening
HCV RNA and antibody positive
HbsAg negative
Hepatitis virus antibody
Liver biopsy within the last 2 years showing features of chronic hepatitis C with cirrhosis
Normal thyroid function tests
Serum ferritin with 2× upper limit of normal
Normal serum albumin
Normal prothrombin index
Refractory to, unwilling or unable to take interferon/ribavarin therapy
Showing symptoms typically associated with chronic hepatitis infection

Subjects who met any of the following criteria at screening were excluded from study participation:
Ongoing chronic illness requiring regular medication including diabetes, hypertension and asthma
Administration of antiviral therapy within the 6 months prior to screening
Concurrent administration of any other investigational drug or participation in a research study within the last three months
Sexually active females not employing reliable contraceptive methods
Pregnant women and nursing mothers
Clinically significant abnormal haematological or biochemical parameters other than those associated with hepatitis.

The use of any new medication, other than study medication, is to be avoided where possible. All concomitant medication taken from 30 days prior to screening until the follow up visit, was recorded.

The primary efficacy parameter was:
1. A Significant reduction of 2 log in serum viral load

The secondary efficacy parameters were:
1. Reduction in liver enzymes
2. Global assessment by subject and investigator

Results:

### Interim Results

The interim results were obtained on the first 9 patients in the methylene blue trial, out of 36 patients enrolled in total. The results showed 33% with a greater than or equal to two log reduction in viral load at 50 days of treatment; and 89% with between greater than or equal to 0.6 log to less than or equal to one 1 log reduction in viral load at 50 days of treatment. This indicates that 8 of 9 patients showed a drop in viral load of between 62%-100% within the first 50 days.

Results At 50 Days

23 patients had a decrease in viral count of between 70-100%. Two patients were non-responders at 50 days.

Of the responders:
12 (52%) had between 0.7-1 log reduction in viral load
6 (26%) had between 1-2 log reduction in viral load
5 (22%) had viral clearance

The results indicate that the methylene blue is highly efficacious in treating hepatitis C.

Modifications and variations of the method to selectively, and in a controlled manner, inhibit specific viruses such as hepatitis virus, and use thereof in the treatment of viral infections will be obvious to those skilled in the art from the foregoing detailed description. Such modifications and variations are intended to come within the scope of the appended claims.

We claim:

1. A method for treating hepatitis virus in a patient comprising:
administering over a period of at lease one month to the patient an effective amount of a thiazine dye having the chemical formula shown below: 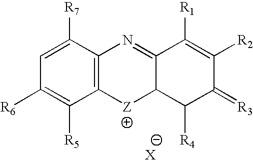
wherein R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen; R₃ and R₆ are independently selected from the group consisting of —O. —NH₂, —NHR₈, and —NR₉R₁₀ and combinations thereof wherein R₈—R₁₀ is a linear, branched or cyclic hydrocarbon or R₉ and R₁₀ together with the nitrogen atom to which they are attached form an optionally substituted 5-, 6-, or 7-membered ring; wherein X⁻is a counterion and wherein Z is either S or O.

2. The method of claim 1 wherein the thiazine dye is selected from the group consisting of methyl methylene blue, dimethyl methylene blue, azure A, azure B, azure C, methylene green, new methylene blue, Taylor's Blue, Toluidine Blue O, thionine and Nile blue.

3. The method of claim 1, wherein the thiazine dye is methylene blue.

4. The method of claim 1 wherein the thiazine dye is a pharmaceutically acceptable salt of methylene blue or a derivative of methylene blue selected from the group consisting of acetate, propionate, succinate, glycolate, lactate, malate, tartarate, citrate, ascorbate, pamoate, maleate, hydroxymaleate, phenylacetate, glutamate, benzoate, salicylate, sulfanilate, 2-acetoxybenzoate, fumarate, tolunesulfonate, naphthalenesulfonate, methanesulfonate, ethane disulfonate, oxalate, and isethionate salts.

5. The method of claim 1 wherein the composition further comprises one or more pharmaceutically acceptable excipients selected from the group consisting of diluents, binders, plasticizers, lubricants, disintegrants, colorants, stabilizers, surfactants, and combinations thereof.

6. The method of claim 1 wherein the composition is administered parenterally as a sterile formulation.

7. The method of claim 1 wherein the composition is formulated for controlled release.

8. The method of claim 1 wherein the dye is administered orally.

9. The method of claim 1 further comprising enhancing the anti-viral activity of the dye by exposure to non-ionizing radiation.

10. The method of claim 1 further comprising one or more therapeutic, prophylactic or diagnostic agents.

11. The method of claim 11 wherein the agent is selected from the group consisting of antibiotics, anti-inflammatories, antifungals, and antivirals.

12. The method of claim 1 wherein the patient is treated for at least two months.

13. A method for decreasing or preventing reactivation of a virus in a patient comprising:
administering to an individual having, or suspected of having, a latent viral infection for an effective period of time an effective amount of a thiazine having the chemical formula shown below: 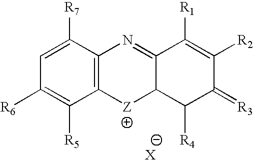
wherein R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen: R₃ and R₆ are independently selected from the group consisting of —O, —NH₂, —NHR₈ , and —NR₉R₁₀ and combinations thereof wherein R₈—R₁₀ is a linear, branched or cyclic hydrocarbon or R₉ and R₁₀ together with the nitrogen atom to which they are attached form an optionally substituted 5-, 6-, or 7-membered ring; wherein X⁻ is a counterion and wherein Z is either S or O.

14. The method of claim 13 wherein the thiazine dye is selected from the group consisting of methyl methylene blue, dimethyl methylene blue, azure A, azure B, azure C, methylene green, new methylene blue, Taylor's Blue, Toluidine Blue O, thionine and Nile blue.

15. The method of claim 13 wherein the thiazine dye is a salt selected from the group consisting of acetate, propionate, succinate, glycolate, lactate, malate, tartarate, citrate, ascorbate, pamoate, maleate, hydroxymaleate, phenylacetate, glutamate, benzoate, salicylate, sulfanilate, 2- acetoxybenzoate, fumarate, tolunesulfonate, naphthalenesulfonate, methanesulfonate, ethane disulfonate, oxalate, and isethionate salts.

16. The method of claim 13 comprising administering methylene blue or a derivative or salt thereof.

17. The method of claim 13 wherein the composition further comprises one or more pharmaceutically acceptable excipients selected from the group consisting of diluents, binders, plasticizers, lubricants, disintegrants, colorants, stabilizers, surfactants, and combinations thereof.

18. The method of claim 13 wherein the composition is administered parenterally as a sterile formulation.

19. The method of claim 13 wherein the composition is formulated for controlled release.

20. The method of claim 13 further comprising enhancing the anti-viral activity of the dye by exposure to non-ionizing radiation.

21. The method of claim 13 further comprising administering one or more therapeutic, prophylactic or diagnostic agents.

22. The method of claim 21 wherein the agent is selected from the group consisting of antibiotics, anti-inflammatories, antifungals, and antivirals.

23. The method of claim 13 wherein the individual is a cancer patient that is or will be undergoing chemotherapy.

24. The method of claim 13 wherein the individual is or will be immunosuppressed.

25. The method of claim 13 wherein the virus is selected from the group consisting of Herpes simplex virus type 1, herpes simplex virus type 2, Varicella zoster virus, Epstein-Barr virus, Cytomegalovirus, and Herpes virus type six, herpes virus type 7, Adenovirus, and Human polyoma viruses.

26. A pharmaceutical composition for administration comprising an effective amount of a derivative or salt of methylene blue, in a pharmaceutically acceptable carrier in a unit dosage form, to prevent a viral infection, inhibit viral replication, or prevent viral reactivation.

27. The composition of claim 26 wherein the derivative of methylene blue has the chemical formula shown below: 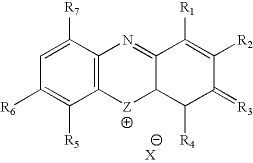
wherein R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen: R₃ and R₆ are independently selected from the group consisting of —O, —NH₂, —NHR₈, and —NR₉R₁₀ and combinations thereof wherein R₈—R₁₀ is a linear, branched or cyclic hydrocarbon or R₉ and R₁₋₀ together with the nitrogen atom to which they are attached form an optionally substituted 5-, 6-, 7-membered ring; wherein X⁻ is a counterion and wherein Z is either S or O.

28. The composition of claim 26 wherein the derivative of methylene blue is selected from the group consisting of methyl methylene blue, dimethyl methylene blue, azure A, azure B, azure C, methylene green, new methylene blue, Taylor's Blue, Toluidine Blue O, thionine and Nile blue.

29. The composition of claim 26 wherein the methylene blue is a salt of methylene blue selected from the group consisting of acetate, propionate, succinate, glycolate, lactate, malate, tartarate, citrate, ascorbate, pamoate, maleate, hydroxymaleate, phenylacetate, glutamate, benzoate, salicylate, sulfanilate, 2- acetoxybenzoate, fumarate, tolunesulfonate, naphthalenesulfonate, methanesulfonate, ethane disulfonate, oxalate, and isethionate salts.

30. The composition of claim 26 further comprising one or more pharmaceutically acceptable excipients selected from the group consisting of diluents, binders, plasticizers, lubricants, disintegrants, colorants, stabilizers, surfactants, and combinations thereof.

31. The composition of claim 26 formulated as a sterile formulation for parenteral administration.

32. The composition of claim 26 wherein the composition is formulated for controlled release.

33. The composition of claim 32 wherein the controlled release composition is formulated for delayed release.

34. The composition of claim 32 wherein the controlled release composition is formulated for extended release.

35. The composition of claim 32 wherein the controlled release composition is formulated for pulsatile release.

36. A controlled release methylene blue formulation.
